(19) United States
(12) Patent Application Publication
(10) Pub. No.: US 2024/0090395 A1
(43) Pub. Date: Mar. 21, 2024

(54) METHOD AND SYSTEM FOR POLLINATION

(71) Applicant: POLYBEE PTE. LTD., Singapore (SG)

(72) Inventor: Siddharth Sunil JADHAV SIDDHARTH SUNIL, Singapore (SG)

(73) Assignee: POLYBEE PTE. LTD., Singapore (SG)

(21) Appl. No.: 18/038,264

(22) PCT Filed: Nov. 19, 2021
(86) PCT No.: PCT/SG2021/050714

§ 371 Date: May 23, 2023

(30) Foreign Application Priority Data
Nov. 23, 2020 (SG) 10202011643V

(51) Int. Cl. A01H 1/02; B64U 10/13; B64U 50/39

(52) U.S. Cl. CPC A01H 1/027; B64U 10/13; B64U 50/39; B64U 2101/70; B64U 2201/10

(57) ABSTRACT

A method of performing pollination of a flower of a plant, and s system for performing pollination. The method comprises the steps of providing a device for generating an airflow; positioning the device relative to the plant such that the flower is subjected to the airflow; and dislodging pollen from the flower as a result of vibrations caused by airflow-induced instabilities in the flower.

### FIELD OF INVENTION

The present invention relates broadly to a method and system for pollination.

### BACKGROUND

Any mention and/or discussion of prior art throughout the specification should not be considered, in any way, as an admission that this prior art is well known or forms part of common general knowledge in the field.

Self-fertile crops have hermaphroditic flowers, i.e., both, male (anthers) and female parts (stigma) are on the same flower. While the name “self-pollinating” suggests that such flowers are pollinated independently without any aid, there are nuances. To dislodge the pollen grains from the anthers such that they settle on the stigmas for germination, an external agency is requisite. Honeybees or bumblebees, if available, are able to perform this function. However, with climate change, ecological imbalances, and rampant in-breeding of bees for commercial production, there are multiple threats to bee populations. In addition, there has been a steady growth of horticulture in controlled environments, such as greenhouses and vertical farms. Bees need extremely specific atmospheric as well as lighting conditions to operate in protected cropping. There are numerous studies pointing out that bees struggle to orientate in protected cropping when lighting and climate is suboptimal for foraging [1, 2]. In such scenarios, pollination is either left to chance, or is performed manually.

WIPO application number WO 2020/095290 A1 (Arugga agtech) describes a ground-based plant treatment system comprising dedicated treatment channels for pollination. The treatment channels are configured to induce vibrations at a specific frequency of 100 Hz or higher, on flowers or parts of plants either via contact or in a contactless method using pulsated air flow.

The contactless method of pollination in WO 2020/095290 A1 involves a fluid flow channel on the ground-based plant treatment system that is capable of emitting well-directed air pulse sequence with a predetermined frequency to induce the requisite vibrations on the flower. Such a mechanism requires provision of a sophisticated fluid flow system that is capable of a short rise time for the air pulse delivery, specifically pulsating jet flow in still air with a predetermined frequency of 100 Hz or higher.

Japan patent application JP2019191854A describes a method of contactless pollination using a ground-based ultrasonic focusing device that excites the target flower at its resonant frequency leading to dislodging of pollen. While ultrasonic emission can induce the requisite force and its frequency to dislodge pollen through pressures waves of pre-defined beat frequency, such a setup requires a sizable grid of ultrasonic actuators mounted on a mobile platform as well as a focusing device to direct the waves at the target.

Embodiments of the present invention seek to address at least one of the above problems.

### SUMMARY

In accordance with a first aspect of the present invention, there is provided a method of performing pollination of a flower of a plant, the method comprising the steps of:
providing a device for generating an airflow;
positioning the device relative to the plant such that the flower is subjected to the airflow; and
dislodging pollen from the flower as a result of vibrations caused by airflow-induced instabilities in the flower.

In accordance with a second aspect of the present invention, there is provided a system for performing pollination, the system comprising:
a control station; and
one or more devices for generating an airflow, the devices being coupled to the control station;
wherein the control station is configured for positioning the one or more devices relative to one or more flowers such that the one or more flowers are subjected to the airflow and for dislodging pollen from the one or more flowers as a result of vibrations caused by airflow-induced instabilities in the one or more flowers.

### BRIEF DESCRIPTION OF THE DRAWINGS

Embodiments of the invention will be better understood and readily apparent to one of ordinary skill in the art from the following written description, by way of example only, and in conjunction with the drawings, in which:

FIG. 1 shows a generic representation of the reproductive parts of a hermaphroditic flower. Aerodynamically controlled pollination enables release of pollen grains once the anther sacs (bottom right) open up after attaining maturity. After depositing on the stigma, the viable pollen grains germinate, and a pollen tube begins to grow (for each pollen grain) via the style towards the ovary for fertilization (image adapted from Wikimedia Commons under C.C.B.Y. 4.0 license).

FIG. 2 shows a photograph illustrating a micro-drone performing aerodynamically controlled pollination (ACP) of a strawberry flower, according to an example embodiment. The drone autonomously navigates over the flower, and subjects it to vibration through its wake. As a result, forces exerted on the flower by the wake dislodge the pollen grains from the anthers to the stigmas.

FIG. 3 shows a photograph illustrating validation of how stigmas turning dark can be used as a marker for successful pollination. The right half of the center of the flower was manually pollinated. The pollination was confirmed by counting the stigmas with the presence of pollen grains on them. In 24 hours, the pollinating stigmas on the right half turned dark.

FIG. 4(a) shows a “before” photograph for illustrating ACP performed on a strawberry flower by autonomous micro-drones, specifically showing the stigmas surrounded by dehisced anthers. The pollen grains are seen as small beads bursting out of the sacs on the anther. This image was taken before the treatment of ACP. Therefore, the stigmas are un-pollinated: i.e. generally clear, without any pollen grains on them.

FIG. 4(b) shows an “after” photograph for illustrating ACP performed on a strawberry flower by autonomous micro-drones, specifically this image was taken after treating the flower with ACP. On closer observation, it is noted that the pollen grains are dislodged from the anthers. There are several stigmas with clearly visible pollen grains on them.

FIG. 5(a) shows a chart illustrating a pollination results for a negative control (random pollination).

FIG. 5(b) shows a chart illustrating pollination results for ACP according to an example embodiment.

FIG. 6 shows a photograph illustrating ACP performed by autonomous micro-drone on tomato flowers in an indoor environment, according to an example embodiment.

FIG. 7 shows a schematic drawing illustrating a platform for ACP according to an example embodiment.

FIG. 8 shows a schematic drawing illustrating a fleet of drones in respective working bays across a farming facility in a platform for ACP according to an example embodiment.

FIG. 9 shows a flowchart illustrating an operating procedure to configure the pollination system according to an example embodiment.

FIG. 10 shows a process chart illustrating a motion planning algorithm/system according to an example embodiment.

FIG. 11 shows a process chart illustrating aspects of the motion planning algorithm/system of FIG. 10.

FIG. 12 shows a process chart illustrating aspects of the motion planning algorithm/system of FIG. 10.

FIG. 13 shows a process chart illustrating aspects of the motion planning algorithm/system of FIG. 10.

FIG. 14 shows a process chart illustrating a motion planning algorithm/system according another example embodiment.

FIG. 15 shows a process chart illustrating aspects of the motion planning algorithm/system of FIG. 14.

FIG. 16 shows a schematic diagram illustrating a drone docketing station with battery swapping mechanism, according to an example embodiment.

FIG. 17 shows a schematic drawing illustrating a land-based system for ACP according to an example embodiment.

FIG. 18 shows a flow-chart illustrating a method of performing pollination of a flower of a plant, according to an example embodiment.

### DETAILED DESCRIPTION

An example embodiment of the present invention provides method of pollination, also referred to herein as aerodynamically controlled pollination (ACP) for self-fertile or self-pollinating crops. These crops include but are not limited to strawberry, raspberry, blueberry, tomato, eggplant, pepper, chili, and okra and represent a sizable proportion of the global agricultural produce.

ACP according to an example embodiment can advantageously satisfy three main requirements for cultivation: high precision, high throughput, and high pollination success rate. In an example embodiment, airflow-induced vibration, instead of pulsating jet flow in still air, is utilized to transfer energy to the flower that maximizes pollen dispersal, and hence, the success rate of pollination. According to various example embodiments, the parameters of the airflow, primarily the Reynolds number, is exploited, as opposed to pulsating jet flow in still air are identified and applied to induce optimal pollen dispersal in the shortest possible time for self-fertile flowers. In one embodiment, airflow that induces appropriate vibrations of the flower is generated by the propellers of autonomous aerial vehicles, preferably micro-drones. The flowers vibrate as they are subjected to the airflow from the drone's downwash as it hovers on the top. These vibrations result in their pollination.

In an example embodiment high-frequency velocity perturbations of airflow are exploited, as opposed to pulsating jet flow in still air. Vibrations are induced in the target flower by the airflow-induced instabilities as a result of the fluid-structure interaction between the airflow (i.e. fluid) and the flower (i.e. structure). These instabilities are of two main types: turbulent, and vortex-induced. In one embodiment, the size of the drone is preferably chosen so as to achieve a specific profile characterized by Reynolds number in its wake that is appropriate for vibration-induced self-pollination across flowers of different morphologies.

In one example embodiment, a method for contactless pollination of self-pollinating crops, also referred to herein as ACP.

In one example embodiment, an autonomous drone capable of flight in indoor environments is provided, preferably a micro-drone.

Aerodynamically Controlled Pollination (ACP) According to an Example Embodiment

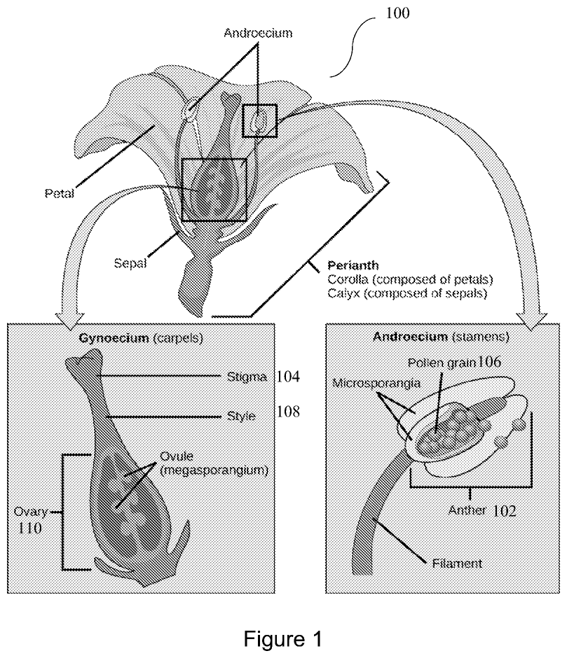

Self-pollinating, or hermaphroditic flowers 100, as illustrated in FIG. 1, are characterized by the presence of both, male (anthers 102) and female (stigma 104) parts of the flower 100. While the anthers 102 shed pollen grains 106, the sticky stigmas 104 receive the viable pollen grains 106, and facilitate the growth of the pollen tube through the style 108 to the ovaries 110 which results in fertilization. It is to be noted that every flower has a high number of ovaries 110 (100-300 for tomatoes and strawberries) to be fertilized. The higher the number of pollen grains 106 deposited on stigmas 104, the higher is the likelihood of germination, and thus, fertilization of the ovaries 110. The higher the number of fertilized ovaries 110, the higher is the likelihood of the resultant fruit being well-shaped and uniform. There is a direct, causal relationship between the degree of pollination, i.e., number of stigmas 104 with viable pollen grains 106 on them, and the quality of fruit.

Even though the anthers 102 and stigmas 104 are on the same flower in self-pollinating flowers, an external agency is essential to mechanically transfer the pollen grains 106. In the absence of such an agency, transfer of pollen grains 106 from anthers 102 to stigma 104 may occur due to random environmental factors, such as movement of the plant due to wind. However, such factors are insufficient to ensure commercially viable production of high-quality produce. To be effective, the mechanism for pollination should maximize the number of stigmas 104 that have pollen grains 106 on them.

A vast number of widely consumed fruits and vegetables, including but not limited to tomatoes, eggplant, pepper, strawberries, raspberries, and blueberries are self-pollinating. In some geographies where bees are available commercially, hives are rented to enhance pollination of these crops, be it in the open fields or in protected cropping. In those geographies where bees are unavailable, pollination is either left to chance, or is performed manually by workers. Especially in protected cropping, bees are vulnerable to a variety of factors that may affect the extent of their foraging activity, and consequently, pollination. Some of these factors are
1. Atmospheric conditions (temperature and relative humidity)
2. Natural lighting: intensity and spectrum
3. Diversity of flora in the accessible environment
4. Access to water

Even where bees are available, there is ample evidence for suboptimal pollination in protected cropping due to these factors. In those geographies where bees are not available at all, there is no alternative to manual pollination. Manual pollination methods, however, have proven to be expensive and not as effective as natural pollinators [3].

Based on the premise of uncertainties around effective pollination and its causal relationship with commercial viability, a scalable method for artificial pollination which is adaptable across different morphologies for flowers is desirable.

In terms of pollination, the central objective is to mechanically transfer the pollen grains 106 from the anthers 102 to the stigmas 104 soon after anthesis. To dislodge a high proportion of viable pollen grains 106 from the anthers 102, the flowers 100 may be subjected to a certain peak velocity which provides sufficient momentum to the anthers 102 to scatter the pollen grains 106. An appropriate means to do so, which also preserves the structure of the flower 100, is to subject the flowers 100 to vibrations. Such a method would work not only for flowers with poricidal anthers, which are not easily accessible for foraging insects such as honeybees, but also for open flowers. It is to be noted that pollen release is a function of time duration of vibrations, and the amplitude of velocity and acceleration [5, 6]. The frequency of the vibrations, in itself, only serves as a means to induce the appropriate acceleration of anthers; a certain amplitude of acceleration and velocity can be achieved through multiple values of frequencies. While the order of magnitude of the frequency does have an effect on pollen release, there is no optimal value for a particular combination of flower and bee [7,8].

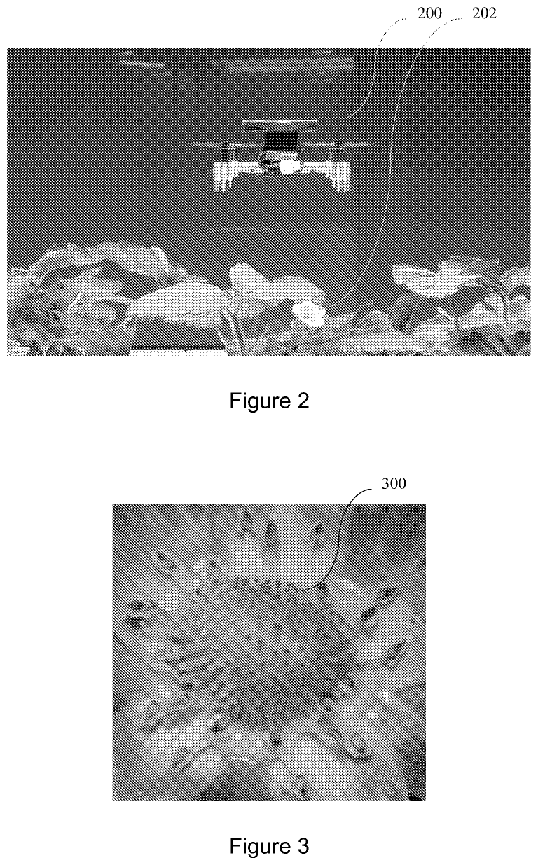

An example embodiment of the present invention provides a low-cost, contactless method of pollination which is agnostic to the morphology of self-pollinating flowers in indoor farming environments where bees cannot generally be used. With reference to FIG. 2, in a method according to an example embodiment, airflow from the downwash of drones 200 is directed at the target flowers 202 ready for pollination. The airflow, while merely being a by-product of the drone 200 flight, eliminates the need to deploy an additional actuator for pollination. This airflow is characterized by time-averaged speed, direction with respect to the orientation of flowers 202, and the Reynolds number for the characteristic length of the flower, typically its height. Those parameters are studied at a distance from the drone 200 where the target flower 202 is expected to be positioned. In an empirical study, drones with varying sizes of propellers, and hence, airflow characteristics, were tested for pollination of strawberries and tomatoes according to various example embodiments. After a few iterations, an example embodiment with suitable specifications for the airflow to pollinate a wide variety of crops such as strawberry, tomato and pepper was provided. In an example embodiment, the airflow is generated by drone propellers of 4.4 cm in diameter, rotating at a speed greater than 15,000 RPM. The flowers were treated with the airflow for about 30 seconds thrice over three days after opening up in testing of an example embodiment.

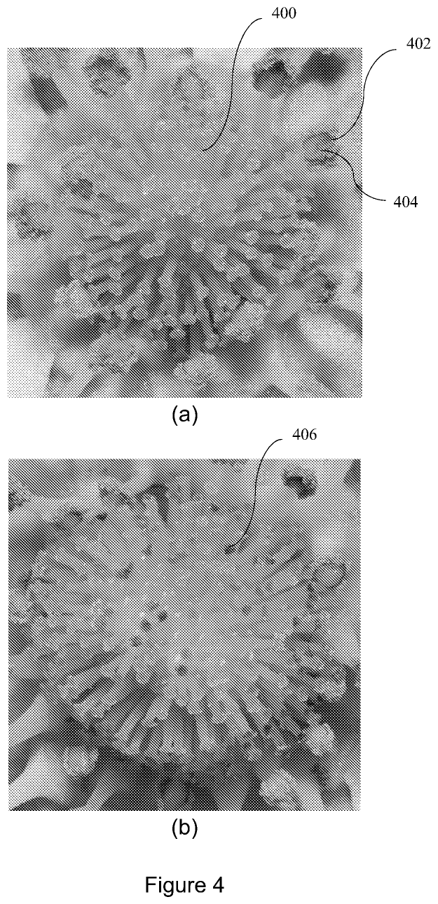

A non-destructive method to evaluate the degree of pollination in strawberry flowers was devised for testing according to an example embodiment. As mentioned, above it is preferred that a high percentage of the total number of stigmas have pollen grains deposited on them to grow high-quality produce. To measure the extent of pollinated stigmas according to an example embodiment, a digital camera with a macro lens was used to image the center of the flower at a high resolution. This enabled to count the number of stigmas with pollen grains on them. While stigmas with pollen grains are clearly visible right after the pollination treatment, another marker for successfully pollinated stigmas was observed. It is conjectured that the stigmas turn dark in roughly 24 hours after successful germination of pollen grains. The validity of using darkened stigmas as an indicator for successful pollination was tested by manually pollinating one half of a strawberry flower. After 24 hours, it was observed that stigmas e.g. 300 of the pollinated half turned dark, as shown in FIG. 3. FIG. 4 illustrates how pollination with ACP according to an example embodiment was evaluated non-destructively using macrophotography. FIG. 4(a) shows a “before” photograph for illustrating ACP performed on a strawberry flower by autonomous micro-drones, specifically showing the stigmas e.g. 400 surrounded by dehisced anthers e.g. 402. The pollen grains e.g. 404 are seen as small beads bursting out of the sacs on the anther 402. This image was taken before the treatment of ACP. Therefore, the stigmas e.g. 400 are un-pollinated: i.e. generally clear, without any pollen grains on them. FIG. 4(b) shows an “after” photograph for illustrating ACP performed on a strawberry flower by autonomous micro-drones, specifically this image was taken after treating the flower with ACP. On closer observation, it is noted that the pollen grains are dislodged from the anthers. There are several stigmas e.g. 406 with clearly visible pollen grains on them.

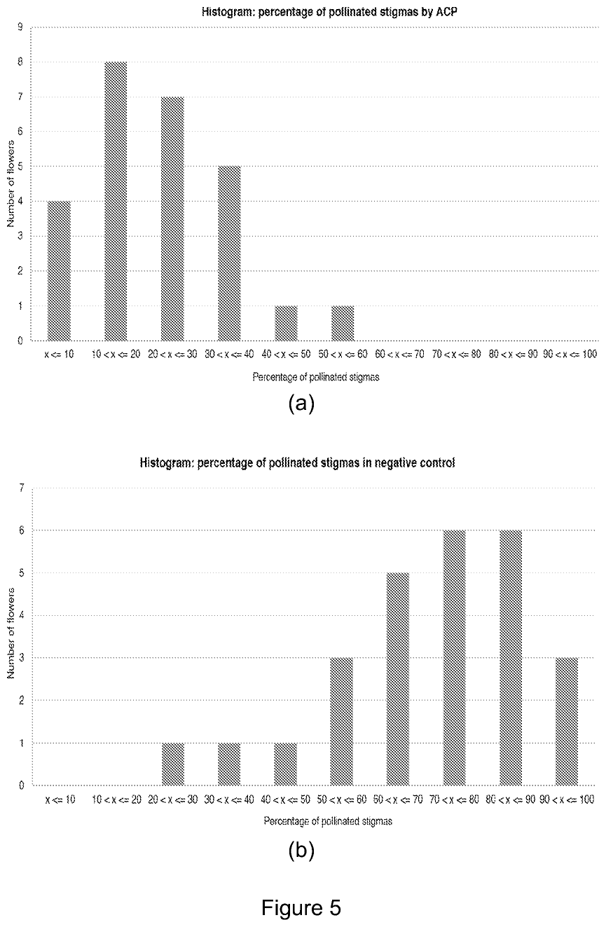

The efficacy of ACP according to an example embodiment was evaluated over a significant number of samples, and was compared to negative control flowers which did not receive any external treatment and were subjected to natural ventilation. While the negative control set had an average of 23% stigmas that were pollinated as shown in FIG. 5(a), the flowers treated with ACP according to an example embodiment had an average of 73% pollinated stigmas as shown in FIG. 5(b). An ANOVA test on the experimental data was run to check for statistical significance. ACP according to an example embodiment was found to have a significant effect on the quality of pollination of strawberries (p<0.0001), where p is the probability that the null hypothesis (method is ineffective) is true, hence the lower the value of p, stronger the evidence for efficacy of ACP according to an example embodiment.

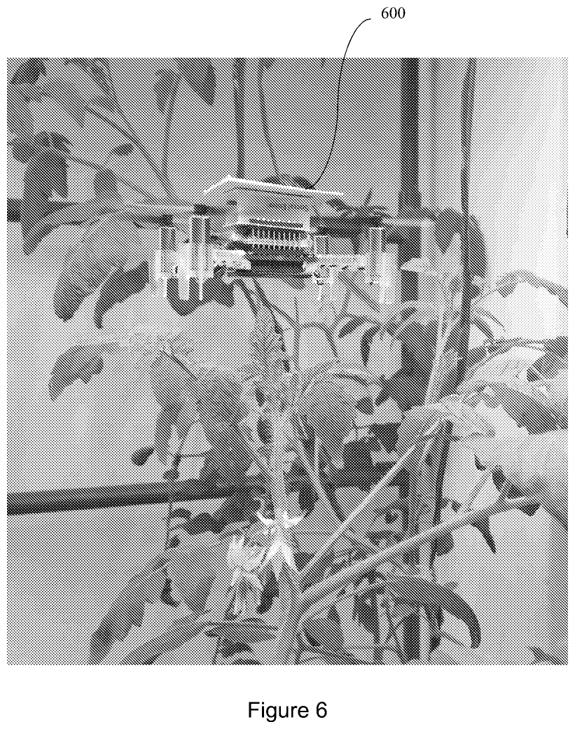

It is noted that the above described method for evaluation of pollination works for strawberries and solanaceous crops with non-poricidal anthers. For solanaceous crops with poricidal anthers, non-destructive early-stage evaluation of pollination is not possible. In tomatoes, the flowers were left to develop fruits after treatment of ACP according to an example embodiment by autonomous micro-drones 600, as illustrated in FIG. 6. A fruit set rate of more than 80% was achieved with ACP according to an example embodiment.

Control and Motion Planning of Autonomous Micro-Drones According to an Example Embodiment

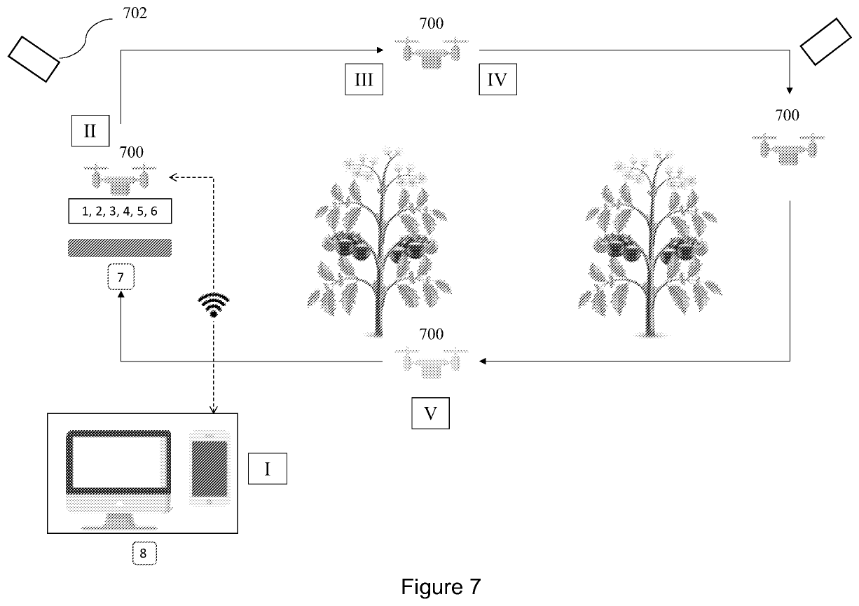

To perform autonomous pollination, a micro-drone 700 platform 701 according to an example embodiment has high-fidelity state estimation. With reference to FIG. 7, this is achieved according to an example embodiment using sensor fusion of external cameras e.g. 702, on-board cameras, e.g. 3D camera with gimbal for sensing indicated at numeral 2 (stereovision), on-board embedded computer with graphics interface indicated at numeral 1, on-board thermal camera indicated at numeral 3, on-board micro-climate sensor indicated at numeral 4, on-board comms module, e.g. WiFi indicated at numeral 5, on-board collision avoidance sensor, e.g. rangefinder and monocular camera indicated at numeral 6, on board inertial measurement unit IMU). drone docking station indicated at numeral 7, and ground control station, e.g. local server/PC/smartphone indicated at numeral 8. While multiple external cameras e.g. 702 provide pose estimation, onboard cameras 3, 6 are used for target detection and mapping the environment to avoid collision, according to a non-limiting example embodiment.

System Configuration and Mission Launch According to an Example Embodiment

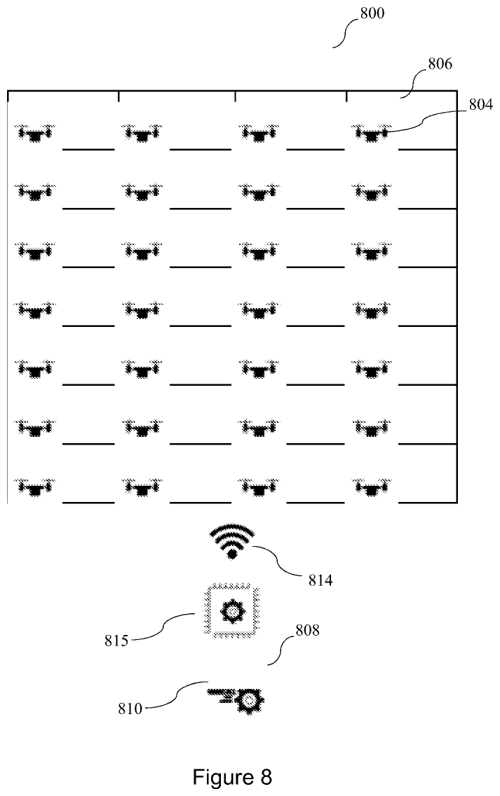

With reference to FIG. 8, the ACP platform or system 800 according to an example embodiment has two main sub-systems: a fleet of autonomous micro-drones e.g. 804, shown in respective working bays e.g. 806 each equipped with a micro-drone e.g. 804 and a docking station (not shown) for re-charging), and a ground control station 808. The communication between the two sub-systems (i.e. fleet of droned e.g. 804, and ground control station 808) is administered by a webapp on a workstation 810 or a phone app through a wireless local network 814. The webapp or phone app, in turn, is connected to a cloud server 815 on the web. The main function of the cloud server 815 is to maintain a database of all operations, host various web services for the phone/webapps, push firmware updates to the drone e.g. 804, and sync up all data collected from the drone e.g. 804.

The first step according to an example embodiment is to configure the co-ordinate system of the environment in which the micro-drone fleet 802 operates, and the calibration of the drone's e.g. 804 state estimation system. The users of the platform 800 according to an example embodiment follow a set of operating procedures to do so. In this procedure, the environment, e.g. a farming facility, is divided into the smaller bays e.g. 806. The co-ordinate system is configured, and the drone e.g. 804 is calibrated by the user on the dashboard running on the ground control station, e.g. workstation 810. The dashboard is also used to initiate, manage and monitor the micro-drone fleet 802 for pollination.

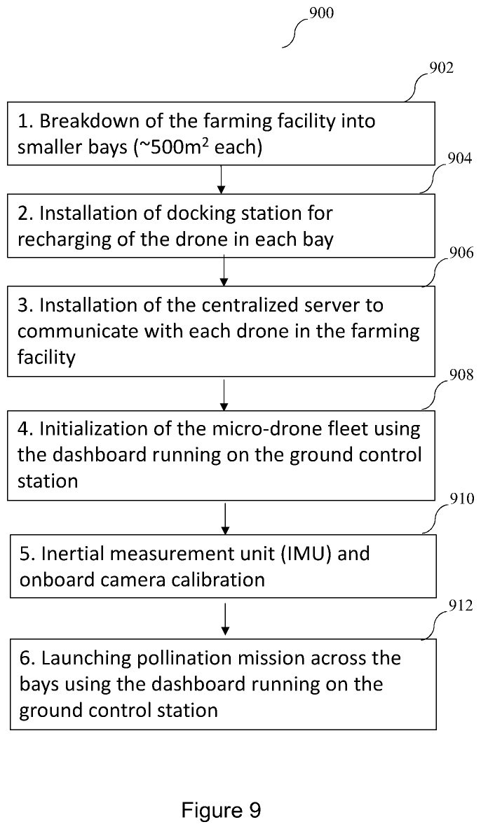

The flow-chart 900 in FIG. 9 elaborates the steps involved in the operating procedure to configure the pollination system according to an example embodiment. At step 902, the farming facility is broken down into the bays (e.g. ˜500 m² each). At step 904, a docking station recharging of the drone is installed in each bay. At step 906, the ground station is installed to communicate with each drone in the farming facility. At step 908, the micro-drone fleet is initialised using the dashboard running on the ground control station. At step 910, the inertial measurement unit (IMU) and onboard cameras are calibrated. In an example embodiment, other on-board components which are off-the-shelf sensors do not need calibration on every reboot. At step 912, a pollination mission is launched across the bays using the dashboard running on the ground control station

Take-Off and Motion Planning According to an Example Embodiment

After the pollination mission is initiated by the user on the ground control station, the drone takes off from its docking station, and runs a motion planning algorithm to find its way to its targets (flowers ready for pollination). The motion planning algorithm can take various forms in different example embodiments. Generally, upon initialization of the pollination system according to an example embodiment through the ground control station, the motion planning algorithm sets the waypoints in the frame of reference of the environment to ensure that the drones fly in close proximity of the targets. For example, the motion planning algorithm according to an example embodiment ensures that the waypoints are set in close proximity of the rows of plants in the farming facility.

In one example embodiment, waypoints are also set for the location of the drone for hovering to perform ACP of a particular plant. Those waypoints may be chosen at a safe height so as to avoid collision with parts of the plant(s).

In another example embodiment, the motion planning algorithm includes functionality for collision avoidance, and local path planning towards target flowers. In such an example embodiment, the onboard sensors (e.g. rangefinders, monocular and/or 3D cameras) detect potential collision objects such as plants and their parts (canopy, stem etc.), structures and objects in the farming facility, and compute the drone's path in real-time on its onboard embedded computer. For local path planning, target detection algorithms are running real-time onboard the drone in such an example embodiment, and are fed by the onboard sensors cameras to identify clusters of flowers ready for pollination. The drone then stabilizes its position to ensure that it is hovering on the top of flower clusters. The waypoints set by the collision avoidance and local path planning towards target flowers override the higher-level path planned by the motion planning algorithm.

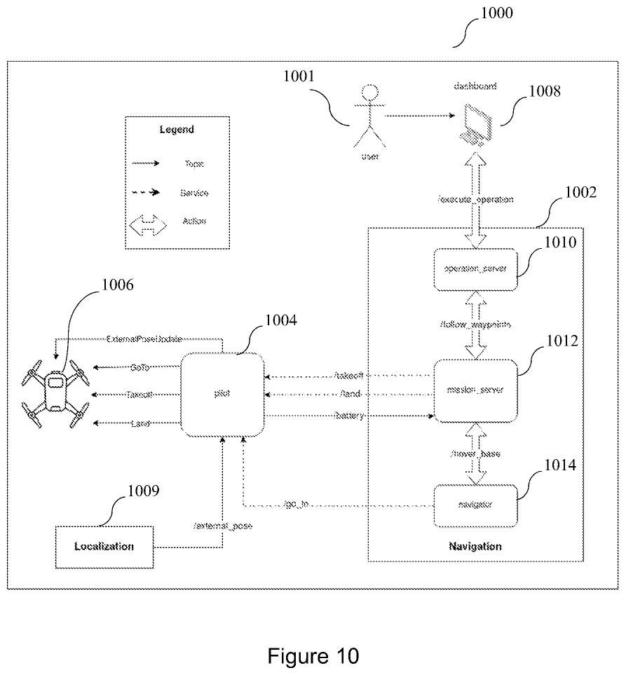

One non-limiting example motion planning algorithm according to an example embodiment will now be described with reference to FIGS. 10 to 13.

In one example embodiment, a relatively “simpler” motion planning algorithms/system is provided, where parts of the algorithm that have the potential to be automated (setting waypoints, obstacle avoidance, etc.) are assumed to be performed manually by the user. This in turn reduces the computational load on the embedded system onboard the drone while also making the core motion planning logic easier to implement and execute. As a limitation, this results in a less dynamic system which does not automatically react to changing environmental conditions making such an example embodiment suitable for an environment that is more or less static or one that changes slowly over time.

With reference to FIG. 10, the motion planning system 1000 according to an example embodiment comprises two main components, the autonomous navigation module 1002 which is responsible for state estimation and motion planning, and the pilot 1004 which is responsible for receiving flight commands from the navigation module 1002 and converting these commands to low-level motor PWM values at the firmware level. The pilot 1004 acts as an interface between the navigation module 1002 and the firmware running on the flight controller onboard the drone 1006. Low-level commands from the pilot 1004 are sent to the firmware using a high-throughput low-latency real time communication protocol., such as CRTP (Crazy RealTime Protocol), I2C (Inter-Integrated Circuit), or MAVLink (Micro Air Vehicle Link).

In the example motion planning system 1000, the web (e.g. via computer dashboard 1008) or phone app can be used by the user 1001 to create an “operation” consisting of a set of “missions” to be carried out. Each mission contains a list of waypoints (points in the 3-dimensional space with reference to an origin point somewhere in the bay the drone is operating in, compare FIG. 8) that represent either a “hover point” or a “pass-through point”. A hover point is defined as a point in space where the drone is expected to hover for a predefined number of seconds, generally a point at an appropriate height above a flower that is to be pollinated using ACP. A pass-through point acts as a checkpoint that the drone 1006 needs to travel through while traveling from one hover point to another. The user-defined list of waypoints is assumed to be collision-free and covering all flowers available in the operational area of the drone that need to be pollinated.

The localization module 1009 in the example motion planning system 1000 is responsible for determining and publishing the location of the drone 1006 with respect to the environment that the drone 1006 is operating in. The localization module 1009 is fashioned to be extremely modular using a plugin-based structure according to an example embodiment. Multiple localization plugins are provided that can be loaded at runtime by changing the system configuration as per the user requirements and environmental dynamics during deployment. The plugins provided within the localization module 1009 according to an example embodiment can include the following:

Fiducial marker-based localization: Fiducial markers offer a cheap alternative to expensive motion capture systems to provide high-frequency localization. In one example embodiment, ArUco markers are used as the choice of fiducial markers for localization. ArUco markers are binary squares with a black background containing a white square pattern that uniquely identifies them. An appropriately sized marker is placed on top of the drone 1006 with downward-facing camera(s) placed strategically in the environment. An image processing algorithm is used on the image stream from the camera(s) to identify the marker such that the size and orientation of the marker in the image can be used to estimate its rotation and translation with respect to the camera, and hence the pose of the drone 1006. In another example embodiment, fiducial markers are placed strategically in the environment and a light-weight wireless camera is placed on top of the drone 1006. The estimated pose of the marker with respect to the camera is used to calculate the pose of the drone 1006 with respect to the environment assuming the position of the camera and markers are known beforehand.

Lighthouse system-based localization: The Lighthouse system uses a lighthouse deck which is mounted on the drone 1006 along with a base station which was originally developed by SteamVR for motion tracking in virtual reality applications. The lighthouse base stations contain a rotating drum which emits infrared (IR) light that is received by four IR receivers available on the lighthouse deck. The relative time taken for the IR light to hit all four receivers along with the angle at which each receiver captures the signal is used to estimate the pose of the drone 1006 with respect to the base station which is placed strategically in the environment.

Visual Inertial Odometry (VIO)-based localization: VIO is defined as the process of pose estimation of an agent using a combination of one or more cameras along with Inertial Measurement Units (IMUs) mounted on the agent. In an example embodiment, a monocular camera is placed on the drone 1006 in addition to the IMU which drones are generally equipped with. Image processing algorithms are used to identify and track relevant features (for example, edges, curves, etc.) in sequential images from the image stream of the camera. The relative movement of tracked features from one image to another combined with the high-frequency inertial data produced by the onboard IMU is used to estimate the rotation and translation of the drone 1006 with respect to the environment.

The navigation module 1002 in this example algorithm comprises three components:

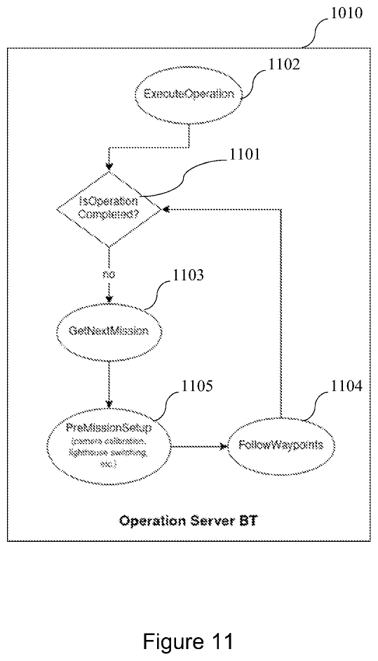

Operation Server 1010: With reference to FIGS. 10 and 11, the operation server 1010 is the point of entry to the navigation module 1002 and is responsible for tracking the state of completion of the ongoing operation, indicated at numeral 1101. The operation server 1010 keeps track of the list of missions that make up the current operation (input at numeral 1102) and is responsible for getting the next mission in the list, indicated at numeral 1103) and sending the next mission to the mission server 1012 once the previous mission has been completed successfully. The interface to the mission server 1012 is indicated at numeral 1104. The operation server 1010 also runs any intermediate tasks between two missions, for example, performing camera calibration, changing lighthouse modes, uploading data to servers, sending crash report to dashboard in case a mission fails, etc., indicated at numeral 1105.

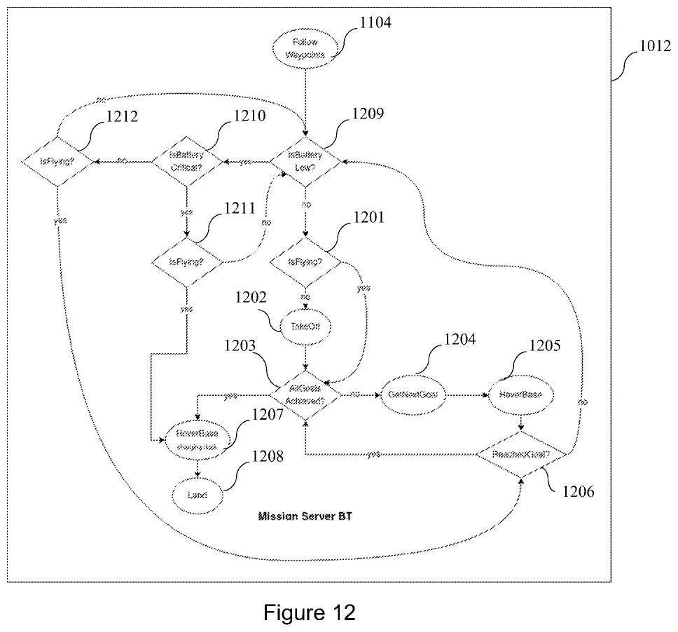

Mission Server 1012: With reference to FIGS. 10 and 12, the mission server 1012 is responsible for tracking the state of completion of the ongoing mission. It keeps track of the list of waypoints from the interface at numeral 1104 to the operation server 1010 that make up the current mission, following the decision/action sequence 1201 to 1208, and is responsible for getting the next waypoint to the navigator 1014 once the previous waypoint has been achieved successfully, indicated at numeral 1204, and sending the next waypoint to the navigator 1014. The interface to the navigator 1014 is indicated at numeral 1205. The mission server 1012 is also responsible for checking the battery level of the drone 1006 periodically, indicated at numeral 1209. The mission server 1012 pauses the ongoing mission and forces the drone 1006 to return to its assigned charging dock if the battery level goes below a predefined threshold value, indicated as the decision/action sequence at numerals 1209 to 1212, 1207, 1208. Once the drone 1006 battery is recharged to an appropriate voltage level, the mission server 1012 commands the drone 1006 to continue the previous mission.

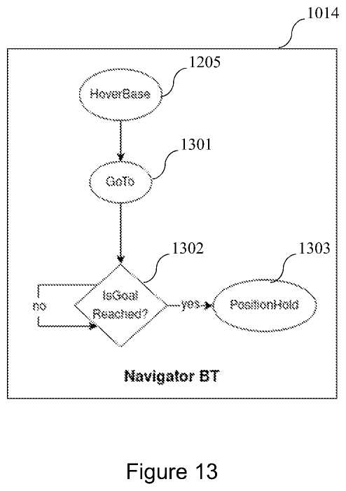

Navigator 1014: With reference to FIGS. 10 and 13, the navigator 1014 is responsible for navigating the drone 1006 from one waypoint to another as received from the interface at numeral 1205 to the mission server 1012, indicated numeral 1301. The navigator 1014 keeps track of the drone's 1006 state estimate and ensures that the drone 1006 successfully reaches within a predefined goal radius around the desired waypoint, indicated at numeral 1302. If the current waypoint happens to be a hover point, the navigator 1014 commands the drone 1006 to hover at the goal for the required number of seconds, indicated at numeral 1303.

Since higher level objectives like pollination and obstacle avoidance are dependent on the quality of the waypoints recorded by the user manually, this motion planning system/algorithm according to an example embodiment does not require a real time mapping module making it much more efficient than a system according to an example embodiment that is more autonomous.

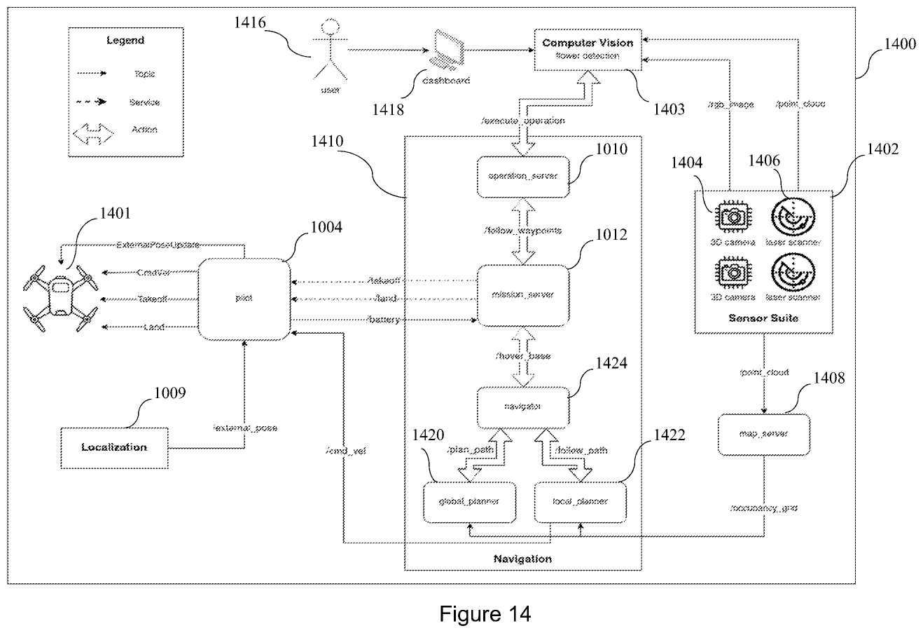

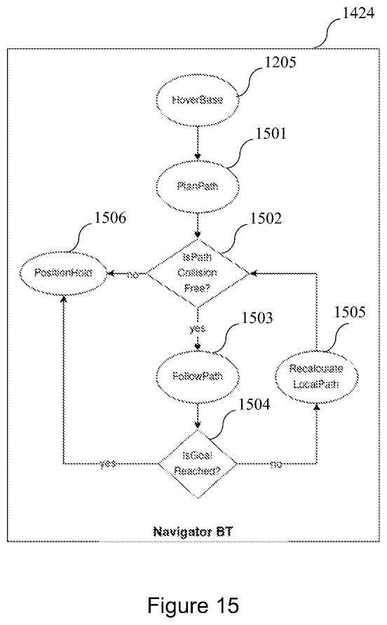

Another non-limiting example motion planning system/algorithm 1400 according to an example embodiment will now be described with reference to FIGS. 14 to 15.

In this example embodiment several sensors are used placed both onboard the drone 1401 and at different strategic locations in the surrounding environment, indicated together at numeral 1402. Information from this sensor suite 1402 is used by the motion planning system 1400 to make informed decisions about where to navigate thus automating the process of waypoint selection in computer vision module 1403 and introducing autonomous navigation capabilities to the system 1400. The sensor suite 1402 includes 3D cameras e.g. 1404 that use stereovision to produce a depth map of the environment while also streaming RGB images. Additionally, laser scanners e.g. 1406 or other types of rangefinder sensors such as sonars, radars, altimeters, etc. can also be used to produce detailed point clouds of the environment. Point cloud data from the available sensors is used by a mapping server/algorithm 1408 to produce a 3D occupancy grid which is used by the navigation module 1410 to add collision-free path planning.

This example embodiment automates the process of creating missions and selecting waypoints by using computer vision module 1403, that harnesses deep neural networks to detect and localize flowers in the bay where the drone 1401 is operating. Once the user 1416 starts the pollination operation using the dashboard on computer 1418 (or via phone app), RGB images streaming from the 3D cameras e.g. 1404 placed in the vicinity are sent to the computer vision module 1403 where a deep convolutional neural network is used to detect flowers. The one-to-one correspondence between the RGB image and the depth map produced by the 3D cameras e.g. 1404 is used to find the 3-dimensional coordinates of each detected flower within the frame of reference of the camera(s). This list of 3D coordinates for all detected flowers is adjusted to be at the appropriate height for the drone 1401 to perform ACP and then sent as an operation to the operation server 1010.

This example embodiment builds on the example embodiment described above with reference to FIGS. 10 to 13 by adding two additional components to the navigation module 1410, namely the global planner 1420 and the local planner 1422 coupled to a modified navigator 1424. The flow of data within the navigation module 1410, starting from the operation server 1010 down to the navigator 1424, is the same as the embodiment described above with reference to FIGS. 10 to 13. The difference between the two example embodiments lies in how the navigator 1424 handles each new waypoint that it receives; the navigator 1424 uses a more complex behaviour tree logic incorporating the global planner 1420 and the local planner 1422.

With reference to FIGS. 14 and 15, the navigator 1424 sends the latest waypoint to the global planner which is responsible for planning a collision-free global path for the drone to follow to reach the waypoint, indicated as the decision/action sequence 1501 to 1506. More specifically, the global planner 1420 uses the 3D occupancy grid produced by the map server using the point clouds produced by the available sensors. The global planner 1420 supports multiple flavours of the Rapidly-exploring Random Tree (RRT) algorithm that efficiently searches for a collision-free path through the 3D occupancy grid by randomly building a space-filling tree.

The global path is in turn sent to the local planner 1422 which is responsible for transforming the global path into smaller segments while taking into consideration the kinematic constraints of the drone 1401 and dynamic obstacles present in the environment. The local planner 1422 works on a much smaller region of the 3D occupancy grid around the drone 1401 in order to efficiently recalculate the path segments without increasing the computational load on the system and thus generates short strategies to avoid dynamic obstacles while trying to remain as close to the global path as possible. The local planner 1422 is effectively the most important component in the navigation module 1410 as it calculates and publishes the lower-level translational commands to the pilot 1004. Once the local planner 1422 successfully guides the drone 1401 to the desired waypoint, indicated at numeral 1506, the navigator informs the upstream mission server 1012 which in turn checks for the successful completion of the current mission and informs its upstream node, the operation server 1010, similar to the upstream flow of data in the example embodiment described above with reference to FIGS. 10 and 13.

Assessing the Conditions for Pollination According to an Example Embodiment

The likelihood of successful germination of pollen grains heavily depends on the local, micro-climatic conditions. The metrics to assess the fitness of conditions are surface temperature of the flower, the ambient air temperature and relative humidity in the vicinity of the plant. Since it is preferred that pollination is attempted under the appropriate micro-climatic conditions, an example embodiment incorporates measurement of micro-climatic conditions through a sensor suite in a feedback loop. If the micro-climatic conditions are not within the optimal window, the micro-drone aborts the pollination visit on the particular cluster of flowers. If the conditions are appropriate, the drone positions itself to hover on the top of the cluster to induce vibrations through its wake.

Pollination Treatment: Aerodynamically Controlled Pollination According to an Example Embodiment

After assessment of the pollination conditions, the drone hovers on the cluster of flowers to induce the pollination treatment. To induce suitable vibrations on the flowers, the main airflow parameter is the Reynolds number of the airflow for the characteristic length of a particular flower, according to an example embodiment. Since the range of the characteristic length of the flowers is typically fixed in a farming facility, the effective Reynolds number depends on the velocity of the airflow in the downwash of the drone. While direction of the velocity is fixed (downward) in an example embodiment, its magnitude is determined by the following factors:
1. Diameter of the propellers
2. Rotational speed of the propellers
3. The distance between the drone and the clusters of flowers

While the diameter of the propellers and the range of their rotational speed is typically fixed for a particular design of the micro-drone, the effective Reynolds number can be varied by adjusting the distance between the drone and clusters of the flower according to an example embodiment. In relation to the diameter of the propellers two drone designs according to non-limiting example embodiments have 9 cm and 15 cm diameter of the propellers, respectively.

Based on empirical studies performed, Reynolds number in a range from about 1×10³ to 1×10⁶, preferably from about 1×10⁴ to 5×10⁵, most preferably from about 1×10⁴ to 5×10⁴ was found to induce the appropriate motion which, in turn, maximizes the likelihood of successful pollination of flowers such as strawberry, blackberry, raspberry, tomato, pepper, chili, and eggplant.

Specifically, this translates into the following setting in the non-limiting example embodiments:

### Drone Embodiment 1

1. Diameter of the propellers: 9 cm
2. Rotational speed of the propellers: 15000 to 20000 RPM
3. The distance between the drone and the clusters of flowers: 15 cm to 1 m

### Drone Embodiment 1

1. Diameter of the propellers: 15 cm
2. Rotational speed of the propellers: 5000 to 10000 RPM
3. The distance between the drone and the clusters of flowers: 15 cm to 1 m

It was found that the duration to subject this airflow to flowers is in a range from about 5 s to 60 s, preferably from about 10 s to 30 s, most preferably from about 10 to 15 seconds, depending on the species, according to an example embodiment. The number of visitations for pollination range from about 1 to 20, preferably from about 2 to 10, most preferably from about 3 to 5 depending on the species, according to an example embodiment.

As described above, in order to dislodge pollen grains from the flower, the mechanism exploited in ACP according to an example embodiment is vibration of the flower caused by the airflow-induced instabilities as a result of the fluid-structure interaction between the airflow (i.e. fluid) and the flower (i.e. structure). These instabilities are of two main types: turbulent, and vortex-induced. It is the Reynolds number of this airflow for the characteristic length of the flower that governs the nature of the vibrations caused by the airflow-induced instabilities. In contrast, a mechanism that is based on pulsating jet flow in still air exploits the momentum impulse of the pulsating jet in still air to cause the vibration of the flowers, and not airflow-induced instabilities. As such, the Reynolds number is a non-factor/irrelevant for a pulsating jet flow mechanism. It is noted that the necessary equipment to create such pulsating jets in still air is significantly more expensive than the cost of goods for a device such as micro-drones that exploit airflow-induced instabilities to cause vibrations instead, according to an example embodiment.

Autonomous Docking and Charging According to an Example Embodiment

The micro-drone fleet according to an example embodiment is preferably designed to function fully autonomously and perpetually. In an example embodiment, when a micro-drones' battery is at about 25% charge, the global planner autonomously plans a path back to the docking station where the micro-drone can recharge via contact or wireless methods. The docking station may also be equipped with a battery swapping mechanism according to an example embodiment to cut down on the turnaround time, and maximize the utilization of the optimal time window for pollination.

Wireless charging according to an example embodiment:

An existing standard for wireless charging can be adopted to charge the drones as they land on the docking station. For instance, the Qi standard of wireless charging, widely used for low-power consumer electronics such as smartphones, can support a charging speed/power of up to 15 W. While such a speed may be feasible for drones <50 g in weight, it may take hours to recharge drones that weigh up to 250 g. The Ki Cordless Kitchen Standard, which is currently under development, could be adopted for the docking station to charge drones that require batteries with a higher power rating.

Battery Swapping According to an Example Embodiment:

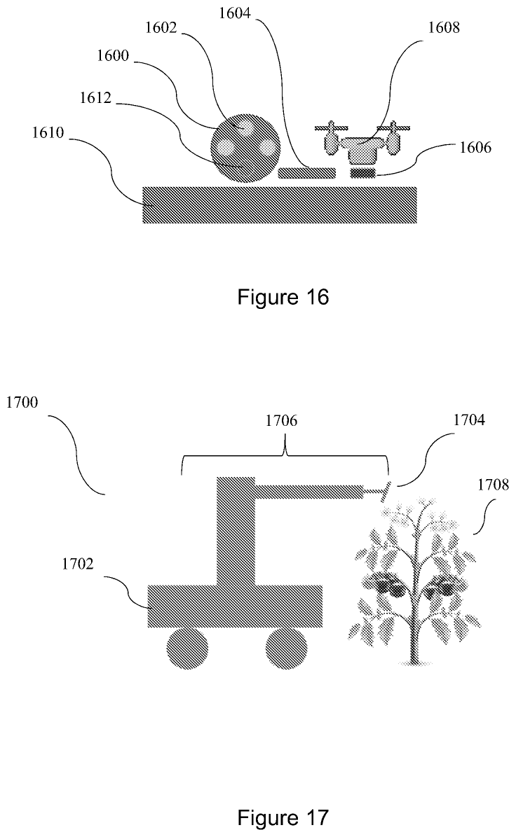

Since high-speed wireless charging standards are yet to be deployed in commercially available products, a battery swapping mechanism is designed according to an example embodiment to mitigate downtime for recharging and maximize availability of drones within time-critical pollination windows. With reference to FIG. 16, the swapping mechanism according to an example embodiment has two main components:
1. Rotating barrel 1600 containing charged batteries e.g. 1602
2. Traversing arm 1604 to retrieve discharged battery 1606 from the drone 1608

The swapping mechanism is triggered after the drone 1608 lands successfully on the docking station 1610. The docking station 1610 is equipped with the rotating barrel 1600 that houses batteries in individual compartments. At any given time, at least one compartment e.g. 1612 is vacant to collect the discharged battery 1606 from the drone using the traversing arm 1604. The arm 1604 places the discharged battery 1606 in the vacant compartment 1612. The rotating barrel 1600 is configured for recharging the discharged battery 1606 via integrated electrical connections coupled to a power supply, implemented for example by a power cable connection of the rotating barrel to a mains outlet. After successful placement of the discharged battery 1606 in the compartment, the barrel 1600 rotates by a certain angle such that a fully charged battery e.g. 1602 can be retrieved by the traversing arm 1604 and transported coupled to the drone 1608.

Pruning of Reductant Flowers According to an Example Embodiment

High robustness in recovery from collisions, for example through a neural-network based control policy according to a preferred embodiment, advantageously provides the drones with a highly valuable capability: pruning of redundant flowers using propellers. In controlled environment cultivation, often, the practices in cultivation are tailored for high quality fruits. This implies that only a stipulated number of fruits are grown on every plant. For instance, the stipulated number can be 5 fruits per truss tomato, and roughly 12 to 14 cherry tomatoes per plant in greenhouses. To ensure this precision, any additional flowers have to be left out of pollination, or even pruned to focus the resources of the plant on the specific flowers. With robust feedback control, the propellers of the drones can be exploited to prune redundant flowers by physically contacting them. While such an action may be seen to be counter-productive with traditional methods of feedback control, the neural network-based controlled policy according to a preferred embodiment can be trained for recovery from such a stimulus.

Modifications in Drone-Based ACP Example Embodiments

There can be circumstances wherein the drone is unable to hover above target flowers due to obstruction from parts of the plant or external objects in the growing space. This can be mitigated according to an example embodiment by have an actuator onboard the drone, i.e., a dedicated airflow actuator, for example rotating blades with at least one degree of freedom, for inducing the requisite vibrations for pollination while the drone's propellers are used to hover at a desired location relative to the plant. In such an example embodiment, instead of the drone hovering on the top of the flower, the drone can be hovering at any other position facing the flower, with the dedicated airflow actuator pointing towards the flower.

Ground-Based ACP According to an Example Embodiment

While the downwash from the propellers of the drone is used to induce vibration for pollination in the example embodiments described above, other platforms can also be equipped with rotating blades that are custom designed to induce the airflow according to other example embodiments. With reference to FIG. 17, in one example embodiment, a robotic platform 1700 is based on one or more autonomous or remotely driven ground vehicles 1702. The rotating blades 1704 in such an example embodiment are coupled to a manipulator 1706 with up to 6 degrees of freedom. The platform 1700 can e.g. be adopted for crops 1708 and environments where the distribution of flowers is not favourable for drone-based ACP.

It is noted that in an example embodiment, a mixture of drones and ground-based devices may be employed for ACP.

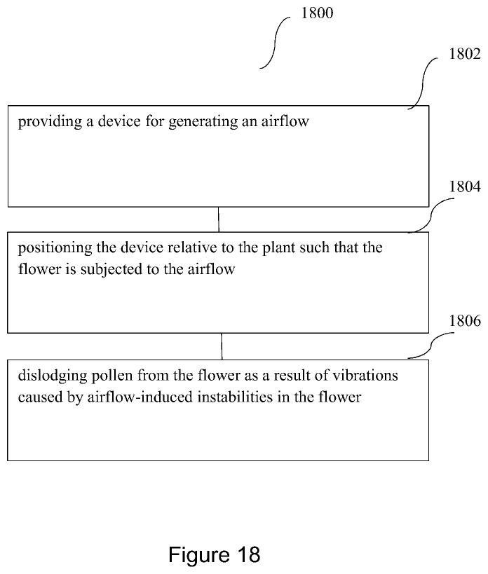

FIG. 18 shows a flow-chart 1800 illustrating a method of performing pollination of a flower of a plant. At step 1802, a device for generating an airflow is provided. At step 1804, the device is positioned relative to the plant such that the flower is subjected to the airflow. At step 1806, pollen is dislodged from the flower as a result of vibrations caused by airflow-induced instabilities in the flower.

The airflow may exhibit a Reynolds number in a range from about 1×10³ to 1×10⁶, preferably from about 1×10⁴ to 5×10⁵, most preferably from about 1×10⁴ to 5×10⁴.

The flower may be subjected to the airflow for a predetermined time period. The time period may be in a range from about 5 s to 60 s, preferably from about 10 s to 30 s, most preferably from about 10 to 15 seconds.

The flower may be subjected to the airflow for a number of times, wherein each time the flower is subjected to the airflow for the predetermined time period. The number of times may be in a range from about 1 to 20, preferably from about 2 to 10, most preferably from about 3 to times.

The device for generating the airflow may comprise a drone. Positioning the drone relative to the plant such that the flower is subjected to the airflow may comprise positioning the drone such that the flower is disposed within a downwash of the propellers of the drone.

The drone may comprise one or more dedicated propellers, as opposed to the propellers providing lift to the drone, for generating the airflow. Positioning the drone relative to the plant such that the flower is subjected to the airflow may comprise manipulating the one or more dedicated propellers for directing the airflow towards the flower.

The device for generating the airflow may comprise a ground-based device. The ground-based device may comprise a vehicle.

The land-based device may comprise one or more dedicated propellers for generating the airflow. Positioning the ground-based device relative to the plant such that the flower is subjected to the airflow may comprise manipulating the one or more dedicated propellers for directing the airflow towards the flower.

The method may comprise using a plurality of the devices within a farming facility.

The device may be configured for autonomous performance of the pollination.

The method may comprise re-charging the device upon detection of a remaining charge threshold. Re-charging the device may comprise swapping a battery of the device.

In one embodiment, a system for performing pollination is provided, the system comprising a control station; and one or more devices for generating an airflow, the devices being coupled to the control station; wherein the control station is configured for positioning the one or more devices relative to one or more flowers such that the one or more flowers are subjected to the airflow and for dislodging pollen from the one or more flowers as a result of vibrations caused by airflow-induced instabilities in the one or more flowers.

The control station may be configured for controlling the airflow to exhibit a Reynolds number in a range from about 1×10³ to 1×10⁶, preferably from about 1×10⁴ to 5×10⁵, most preferably from about 1×10⁴ to 5×10⁴.

The control station may be configured for subjecting the one or more flowers to the airflow for a predetermined time period. The time period may be in a range from about 5 s to 60 s, preferably from about 10 s to 30 s, most preferably from about 10 to 15 seconds.

The control station may be configured for subjecting the one or more flowers to the airflow for a number of times, wherein each time the one or more flowers are subjected to the airflow for the predetermined time period. The number of times may be in a range from about 1 to 20, preferably from about 2 to 10, most preferably from about 3 to 5 times.

The one or more devices for generating the airflow may comprise one or more drones. The control station may be configured such that positioning the one or more drones relative to the one or more flowers comprises positioning the one or more drones such that the one or more flowers are disposed within a downwash of the propellers of the one or more drones.

Each drone may comprise one or more dedicated propellers, as opposed to the propellers providing lift to the drone, for generating the airflow. The control station may be configured such that positioning the one or more drones relative to the one or more flowers comprises manipulating the one or more dedicated propellers of the one or more drones for directing the airflow towards the one or more flowers.

The one or more devices for generating the airflow may comprise one or more ground-based devices. The one or more ground-based devices may comprise one or more vehicles.

Each ground-based device may comprise one or more dedicated propellers for generating the airflow. The control station may be configured such that positioning the one or more ground-based devices relative to the plant comprises manipulating the one or more dedicated propellers of the one or more ground-based devices for directing the airflow towards the flower.

The one or more devices may be configured for autonomous performance of the pollination.

The system may comprise a detection unit for detecting a remaining charge threshold for the one or more devices and a re-charging unit for the one or more devices. The re-charging unit may be configured for swapping a battery of the one or more devices.

Example embodiment can have one or more of the following features and associated benefits/advantages:

| Feature | Benefit/Advantage |
|---|---|
| Fully autonomous | Autonomous drones or land-based robotic |
| actuation for | platforms eliminate manual labor, one of the |
| pollination | biggest sources of operational costs in |
| | industrial farming. |
| Precision in | With end-to-end autonomous capabilities, |
| space and time | flowers can be pollinated at just the right |
| | time, and on a one-by-one basis to enhance |
| | the quantity as well quality of yields without |
| | additional operational costs. |
| Contactless | A contactless method of pollination offers |
| pollination | higher probability of success by significantly |
| | easing the development of autonomous |
| | robotic solutions. Several issues around |
| | spread of infections and pests, and bruising |
| | due to contact are circumvented by a |
| | contactless method. |

### INDUSTRIAL APPLICATION OF EXAMPLE EMBODIMENTS

Embodiments of the present invention can provide a fully autonomous solution for pollination of self-fertile horticultural crops, including but not limited to tomatoes, eggplant, pepper, strawberries, raspberries, and blueberries. The application environments for these solutions can be ranging from commercial growers to plant breeders, including, but not limited to:
1. Greenhouse growers: polytunnel, glasshouse, netting
2. Indoor vertical farms
3. Agriculture companies undertaking plant breeding activities, such as seed companies

The various functions or processes disclosed herein may be described as data and/or instructions embodied in various computer-readable media, in terms of their behavioral, register transfer, logic component, transistor, layout geometries, and/or other characteristics. Computer-readable media in which such formatted data and/or instructions may be embodied include, but are not limited to, non-volatile storage media in various forms (e.g., optical, magnetic or semiconductor storage media) and carrier waves that may be used to transfer such formatted data and/or instructions through wireless, optical, or wired signaling media or any combination thereof. Examples of transfers of such formatted data and/or instructions by carrier waves include, but are not limited to, transfers (uploads, downloads, e-mail, etc.) over the internet and/or other computer networks via one or more data transfer protocols (e.g., HTTP, FTP, SMTP, etc.). When received within a computer system via one or more computer-readable media, such data and/or instruction-based expressions of components and/or processes under the system described may be processed by a processing entity (e.g., one or more processors) within the computer system in conjunction with execution of one or more other computer programs.

Aspects of the systems and methods described herein may be implemented as functionality programmed into any of a variety of circuitry, including programmable logic devices (PLDs), such as field programmable gate arrays (FPGAs), programmable array logic (PAL) devices, electrically programmable logic and memory devices and standard cell-based devices, as well as application specific integrated circuits (ASICs). Some other possibilities for implementing aspects of the system include: microcontrollers with memory (such as electronically erasable programmable read only memory (EEPROM)), embedded microprocessors, firmware, software, etc. Furthermore, aspects of the system may be embodied in microprocessors having software-based circuit emulation, discrete logic (sequential and combinatorial), custom devices, fuzzy (neural) logic, quantum devices, and hybrids of any of the above device types.

Of course the underlying device technologies may be provided in a variety of component types, e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, etc.

The various functions or processes disclosed herein may be described as data and/or instructions embodied in various computer-readable media, in terms of their behavioral, register transfer, logic component, transistor, layout geometries, and/or other characteristics. Computer-readable media in which such formatted data and/or instructions may be embodied include, but are not limited to, non-volatile storage media in various forms (e.g., optical, magnetic or semiconductor storage media) and carrier waves that may be used to transfer such formatted data and/or instructions through wireless, optical, or wired signaling media or any combination thereof. When received into any of a variety of circuitry (e.g. a computer), such data and/or instruction may be processed by a processing entity (e.g., one or more processors).

The above description of illustrated embodiments of the systems and methods is not intended to be exhaustive or to limit the systems and methods to the precise forms disclosed. While specific embodiments of, and examples for, the systems components and methods are described herein for illustrative purposes, various equivalent modifications are possible within the scope of the systems, components and methods, as those skilled in the relevant art will recognize. The teachings of the systems and methods provided herein can be applied to other processing systems and methods, not only for the systems and methods described above.

It will be appreciated by a person skilled in the art that numerous variations and/or modifications may be made to the present invention as shown in the specific embodiments without departing from the spirit or scope of the invention as broadly described. The present embodiments are, therefore, to be considered in all respects to be illustrative and not restrictive. Also, the invention includes any combination of features described for different embodiments, including in the summary section, even if the feature or combination of features is not explicitly specified in the claims or the detailed description of the present embodiments.

In general, in the following claims, the terms used should not be construed to limit the systems and methods to the specific embodiments disclosed in the specification and the claims, but should be construed to include all processing systems that operate under the claims. Accordingly, the systems and methods are not limited by the disclosure, but instead the scope of the systems and methods is to be determined entirely by the claims.

Unless the context clearly requires otherwise, throughout the description and the claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import refer to this application as a whole and not to any particular portions of this application. When the word “or” is used in reference to a list of two or more items, that word covers all of the following interpretations of the word: any of the items in the list, all of the items in the list and any combination of the items in the list.

### REFERENCES

1. L J Evans et al. (2019), “Netted crop covers reduce honeybee foraging activity and colony strength in a mass flowering crop”, Ecology and Evolution.
2. https://www.koppert.com/news/pollination-under-artificial-lights/3.www.aph.gov.au/Parliamentary Business/Committees/Senate/Environment and Communi cations/Bumblebees45/Report/c02
4. Lihoreau M and Raine N E (2013), “Bee positive: the importance of electroreception in pollinator cognitive ecology”. Front. Psychol. 4:445. doi: 10.3389/fpsyg.2013.00445
5. De Luca, P. A., Bussière, L. F., Souto-Vilaros, D. et al. Variability in bumblebee pollination buzzes affects the quantity of pollen released from flowers. Oecologia 172, 805-816 (2013).
6. Vallejo-Marin, M. (2019), Buzz pollination: studying bee vibrations on flowers. New Phytol, 224: 1068-1074.
7. Rosi-Denadai, C. A., Araújo, P. C. S., Campos, L. A. d. O., Cosme, L., Jr. and Guedes, R. N. C. (2020), Buzz-pollination in Neotropical bees: genus-dependent frequencies and lack of optimal frequency for pollen release. Insect Science, 27: 133-142.
8. Tayal, M., Chavana, J. & Kariyat, R. R. Efficiency of using electric toothbrush as an alternative to a tuning fork for artificial buzz pollination is independent of instrument buzzing frequency. BMC Ecol 20, 8 (2020).

1. A method of performing pollination of a flower of a plant, the method comprising the steps of:
providing a device for generating an airflow;
positioning the device relative to the plant such that the flower is subjected to the airflow; and
dislodging pollen from the flower as a result of vibrations caused by airflow-induced instabilities in the flower.

2. The method of claim 1, wherein the airflow exhibits a Reynolds number in a range from about 1×10³ to 1×10⁶, preferably from about 1×10⁴ to 5×10⁵, most preferably from about 1×10⁴ to 5×10⁴.

3. The method of claim 1, wherein the flower is subjected to the airflow for a predetermined time period, and optionally wherein the time period is in a range from about 5 s to 60 s, preferably from about 10 s to 30 s, most preferably from about 10 to 15 seconds.

4. (canceled)

5. The method of claim 3, wherein the flower is subjected to the airflow for a number of times, wherein each time the flower is subjected to the airflow for the predetermined time period, and optionally wherein the number of times is in a range from about 1 to 20, preferably from about 2 to 10, most preferably from about 3 to 5 times.

6. (canceled)

7. The method of claim 1, wherein the device for generating the airflow comprises a drone.

8. The method of claim 7, wherein positioning the drone relative to the plant such that the flower is subjected to the airflow comprises positioning the drone such that the flower is disposed within a downwash of the propellers of the drone, or wherein the drone comprises one or more dedicated propellers, as opposed to the propellers providing lift to the drone, for generating the airflow, optionally wherein positioning the drone relative to the plant such that the flower is subjected to the airflow comprises manipulating the one or more dedicated propellers for directing the airflow towards the flower.

9. (canceled)

10. (canceled)

11. The method of claim 1, wherein the device for generating the airflow comprises a ground-based device, and optionally wherein the ground-based device comprises a vehicle.

12. (canceled)

13. The method of claim 11, wherein the land-based device comprises one or more dedicated propellers for generating the airflow, and optionally wherein positioning the ground-based device relative to the plant such that the flower is subjected to the airflow comprises manipulating the one or more dedicated propellers for directing the airflow towards the flower.

14. (canceled)

15. The method of claim 1, comprising using a plurality of the devices within a farming facility.

16. The method of claim 1, wherein the device is configured for autonomous performance of the pollination.

17. (canceled)

18. (canceled)

19. A system for performing pollination, the system comprising:
a control station; and
one or more devices for generating an airflow, the devices being coupled to the control station;
wherein the control station is configured for positioning the one or more devices relative to one or more flowers such that the one or more flowers are subjected to the airflow and for dislodging pollen from the one or more flowers as a result of vibrations caused by airflow-induced instabilities in the one or more flowers.

20. The system of claim 19, wherein the control station is configured for controlling the airflow to exhibit a Reynolds number in a range from about 1×10³ to 1×10⁶, preferably from about 1×10⁴ to 5×10⁵, most preferably from about 1×10⁴ to 5×10⁴.

21. The system of claim 19, wherein the control station is configured for subjecting the one or more flowers to the airflow for a predetermined time period, and optionally wherein the time period is in a range from about 5 s to 60 s, preferably from about 10 s to 30 s, most preferably from about 10 to 15 seconds.

22. (canceled)

23. The system of claim 21, wherein the control station is configured for subjecting the one or more flowers to the airflow for a number of times, wherein each time the one or more flowers are subjected to the airflow for the predetermined time period, and optionally wherein the number of times is in a range from about 1 to 20, preferably from about 2 to 10, most preferably from about 3 to 5 times.

24. (canceled)

25. The system of claim 19, wherein the one or more devices for generating the airflow comprise one or more drones.

26. The system of claim 25, wherein the control station is configured such that positioning the one or more drones relative to the one or more flowers comprises positioning the one or more drones such that the one or more flowers are disposed within a downwash of the propellers of the one or more drones, or wherein each drone comprises one or more dedicated propellers, as opposed to the propellers providing lift to the drone, for generating the airflow, wherein the control station is configured such that positioning the one or more drones relative to the one or more flowers comprises manipulating the one or more dedicated propellers of the one or more drones for directing the airflow towards the one or more flowers.

27. (canceled)

28. (canceled)

29. The system of claim 19, wherein the one or more devices for generating the airflow comprise one or more ground-based devices, and optionally wherein the one or more ground-based devices comprise one or more vehicles.

30. (canceled)

31. The system of claim 29, wherein each ground-based device comprises one or more dedicated propellers for generating the airflow, and optionally wherein the control station is configured such that positioning the one or more ground-based devices relative to the plant comprises manipulating the one or more dedicated propellers of the one or more ground-based devices for directing the airflow towards the flower.

32. (canceled)

33. The system of claim 19, wherein the one or more devices are configured for autonomous performance of the pollination.

34. The system of claim 19, comprising a detection unit for detecting a remaining charge threshold for the device and a re-charging unit for the one or more devices, and optionally wherein the re-charging unit is configured for swapping a battery of the device.

35. (canceled)
